(19) United States
(12) Patent Application Publication
(10) Pub. No.: US 2017/0112750 A1
(43) Pub. Date: Apr. 27, 2017

(54) ANTIPERSPIRANT COSMETICS COMPRISING SPECIFIC PROTEINS FROM POACEAE OF THE GENUS TRITICUM AND/OR ORYZA AND/OR AVENA AND INCLUDING NO ALUMINUM AND/OR ZIRCONIUM HALIDES AND/OR HYDROXY HALIDES

(71) Applicant: Henkel AG & Co. KGaA, Duesseldorf (DE)

(72) Inventors: Bernhard Banowski, Duesseldorf (DE); Stefan Evers, Haan (DE)

(73) Assignee: Henkel AG & Co. KGaA, Duesseldorf (DE)

(21) Appl. No.: 15/399,673

(22) Filed: Jan. 5, 2017

Related U.S. Application Data
(63) Continuation of application No. PCT/EP2015/064492, filed on Jun. 26, 2015.

(30) Foreign Application Priority Data
Jul. 8, 2014 (DE) 10 2014 213 228.2

(51) Int. Cl. A61K 8/64; A61Q 15/00

(52) U.S. Cl. CPC A61K 8/645; A61Q 15/00; A61K 2800/88; A61K 2800/30

(57) ABSTRACT

The invention relates to an antiperspirant cosmetic including at least one specific protein from poaceae of the genus triticum and/or oryza and including no aluminum and/or zirconium halides and/or hydroxy halides. The invention further relates to the use of a specific protein and to a non-therapeutic method for reducing body perspiration. Adding or using said at least one specific protein ensures that the sweat gland(s) is/are effectively influenced, thus resulting in a significant reduction in axillary hyperhidrosis even in the absence of antiperspirant aluminum salts.

### FIELD OF THE INVENTION

The present invention generally relates to an antiperspirant cosmetic agent without aluminum and/or zirconium halides and/or hydroxy halides which includes at least one substance selected from the group of cosmetic oils that are liquid at 20° C. and 1013 hPa, odorants and waxes, optionally at least one propellant, as well as specific proteins from Poaceae of the genus Triticum and/or Oryza and/or Avena. The addition of the at least one specific protein results in an influencing of the sweat gland(s).

Furthermore, the present invention relates to a packaging unit (kit of parts) including a cosmetic agent according to the invention as well as a cosmetic agent with at least one antiperspirant active substance.

Moreover, the present invention relates to the use of specific proteins from Poaceae of the genus Triticum and/or Oryza and/or Avena for the at least partial influencing of the sweat gland(s).

In addition, the present invention relates to the use of a combination which includes at least one substance selected from the group of cosmetic oils that are liquid at 20° C. and 1013 hPa, odorants and waxes, optionally at least one propellant, as well as specific proteins from Poaceae of the genus Triticum and/or Oryza and/or Avena, to reduce and/or prevent perspiration, particularly underarm perspiration or perspiration in other areas of the body. The combination according to the invention does not include any aluminum and/or zirconium halides and/or hydroxy halides.

Furthermore, the present invention relates to an antiperspirant cosmetic agent without aluminum and/or zirconium halides and/or hydroxy halides which includes at least one substance selected from the group of cosmetic oils that are liquid at 20° C. and 1013 hPa, odorants and waxes, optionally at least one propellant, as well as at least one specific protein isolated from the genus Triticum (wheat). The addition of the at least one specific protein results in an influencing of the sweat gland(s).

Finally, the present invention relates to a non-therapeutic cosmetic method for preventing and/or reducing body odor triggered by perspiration and or body perspiration per se in which an antiperspirant cosmetic agent according to the invention is applied to the skin, particularly to the skin of the underarms, and remains on the skin of the underarms for at least 1 hour, preferably for at least 2 hours, more preferably for at least 4 hours, particularly for at least 6 hours.

### BACKGROUND OF THE INVENTION

The washing, cleansing and nurturing of one's own body represents a basic human need, and modern industry is constantly attempting to meet these human needs in a wide variety of ways. One particularly important aspect of daily hygiene is the lasting elimination or at least reduction of body odor and underarm wetness. Numerous special deodorizing or antiperspirant body care products are known in the prior art that were developed for use in areas of the body with a high density of sweat glands, particularly in the underarm region. These are packaged in an extremely wide variety of presentations, for example as powder, in the form of a pen, as aerosol spray, pump spray, liquid and gel-type roll-on application, cream, gel, and as soaked flexible substrates (deo wipes).

Besides at least one oil or a wax and an odorant component or a perfume, cosmetic antiperspirants of the prior art include at least one antiperspirant compound, particularly in the form of aluminum and/or zirconium halides and/or hydroxy halides. On the one hand, these antiperspirant compounds reduce the secretion of sweat by the body by temporarily narrowing and/or blocking the excretory ducts of the sweat glands, whereby the amount of sweat can be reduced by about 20 to 60 percent. On the other hand, due to their antimicrobial effect, they have an additional deodorizing effect.

In conjunction with the acidic pH of these antiperspirants, aluminum and/or zirconium halides and/or hydroxy halides can result in unpleasant skin reactions in some users. Moreover, the use of the aforementioned antiperspirant compounds can cause staining of clothes.

There is therefore a need to exchange antiperspirant aluminum and/or zirconium halides and/or hydroxy halides for other antiperspirant cosmetic active substances. These antiperspirant active substances are to have a good antiperspirant effect, have good skin compatibility, and be easy to formulate. Furthermore, these antiperspirant active substances are to have no negative impact on the storage stability of the antiperspirant cosmetic agents.

It was therefore the object of the present invention to provide an antiperspirant cosmetic agent that avoids or at least mitigates the drawbacks of the prior art and has good skin compatibility while simultaneously offering reliable reduction of underarm wetness. Moreover, the antiperspirant cosmetic agent is to have a high level of storage stability.

Surprisingly, it was found that the use of at least one protein from Poaceae of the genus Triticum and/or Oryza and/or Avena that brings about a change in the light absorption from 1 to 100% when the pH changes from at least 0.5 to a pH range of pH 4.0 to pH 8.0 in cosmetic agents without antiperspirant aluminum and/or zirconium halides and/or hydroxy halides results in an antiperspirant effect that is nearly equal to the antiperspirant effect of formulations with aluminum salts and/or aluminum-zirconium complexes.

Furthermore, other desirable features and characteristics of the present invention will become apparent from the subsequent detailed description of the invention and the appended claims, taken in conjunction with this background of the invention.

### BRIEF SUMMARY OF THE INVENTION

An antiperspirant cosmetic agent, including at least one substance selected from the group of cosmetic oils that are liquid at 20° C. and 1013 hPa, odorants and waxes; propellant in a total quantity of 0 to 99 wt % with respect to the total weight of the antiperspirant cosmetic agent; and at least one protein in a total quantity of 0.1 to 70 wt % with respect to the total weight of the antiperspirant cosmetic agent, with the at least one protein occurring in Poaceae of the genus Triticum and/or Oryza and/or Avena and with the at least one protein bringing about a change in the light absorption of 1 to 100% when the pH changes from at least 0.5 to a pH range of 4.0 to pH 8.0, a temperature of 20° C. to 40° C. and a concentration of the protein of 0.001 to 10 wt %, with respect to the total weight of the sample mixture used to determine the change in light absorption, with the antiperspirant cosmetic agent not including any aluminum and/or zirconium halides and/or hydroxy halides.

A packaging unit (kit of parts), comprising—packaged separately from one another—at least one first container (C1), including a cosmetic agent (M1) comprising at least one antiperspirant agent; and at least one second container (C2), including a cosmetic agent (M2) comprising at least one protein, with the at least one protein occurring in Poaceae of the genus Triticum and/or Oryza and/or Avena, with the at least one protein bringing about a change in the light absorption of 1 to 100% when the pH changes from at least 0.5 to a pH range of 4.0 to pH 8.0, a temperature of 20° C. to 40° C. and a concentration of the protein of 0.001 to 10 wt %, with respect to the total weight of the sample mixture used to determine the change in light absorption, and with the cosmetic agent (M2) including no aluminum and/or zirconium halides and/or hydroxy halides.

A use of at least one protein for the at least partial influencing of the sweat gland(s), with the at least one protein occurring in Poaceae of the genus Triticum and/or Oryza and/or Avena, and with the at least one protein bringing about a change in the light absorption of 1 to 100% when the pH changes from at least 0.5 to a pH range of pH 4.0 to pH 8.0, a temperature of 20° C. to 40° C., and a concentration of the protein of 0.001 to 10 wt %, with respect to the total weight of the sample mixture used to determine the change in light absorption.

A use of a combination, including at least one substance selected from the group of cosmetic oils that are liquid at 20° C. and 1013 hPa, odorants and waxes; propellant in a total quantity of 0 to 99 wt % with respect to the total weight of the antiperspirant cosmetic agent; and at least one protein in a total quantity of 0.1 to 70 wt % with respect to the total weight of the antiperspirant cosmetic agent, with the at least one protein occurring in Poaceae of the genus Triticum and/or Oryza and/or Avena, with the at least one protein bringing about a change in the light absorption of 1 to 100% when the pH changes from at least 0.5 to a pH range of 4.0 to pH 8.0, a temperature of 20° C. to 40° C. and a concentration of the protein of 0.001 to 10 wt %, with respect to the total weight of the sample mixture used to determine the change in light absorption, and with the combination not including any aluminum and/or zirconium halides and/or hydroxy halides, to reduce and/or prevent perspiration, particularly underarm perspiration or perspiration in other areas of the body.

An antiperspirant cosmetic agent, including at least one substance selected from the group of cosmetic oils that are liquid at 20° C. and 1013 hPa, odorants and waxes; propellant in a total quantity of 0 to 99 wt % with respect to the total weight of the antiperspirant cosmetic agent; and at least one protein isolated from Poaceae of the genus Triticum (wheat) in a total quantity of 0.1 to 70 wt % with respect to the total weight of the antiperspirant cosmetic agent, with the protein being hydrolyzed and/or with the protein being functionalized with at least one cationic group selected from lauryldimonium hydroxypropyl groups, hydroxypropyltrimonium groups and mixtures thereof, with the antiperspirant cosmetic agent not including any aluminum and/or zirconium halides and/or hydroxy halides.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The object of the present invention is an antiperspirant cosmetic agent which includes
a) at least one substance selected from the group of cosmetic oils that are liquid at 20° C. and 1013 hPa, odorants and waxes,
b) propellant in a total quantity of 0 to 99 wt % with respect to the total weight of the antiperspirant cosmetic agent, and
c) at least one protein in a total quantity of 0.1 to 70 wt % with respect to the total weight of the antiperspirant cosmetic agent, with the at least one protein occurring in Poaceae of the genus Triticum and/or Oryza and/or Avena and with the at least one protein bringing about a change in the light absorption of 1 to 100% when the pH changes from at least 0.5 to a pH range of 4.0 to pH 8.0, a temperature of 20° C. to 40° C. and a concentration of the protein of 0.001 to 10 wt %, with respect to the total weight of the sample mixture used to determine the change in light absorption,
   with the antiperspirant cosmetic agent not including any aluminum and/or zirconium halides and/or hydroxy halides.

The use of the at least one protein from Poaceae of the genus Triticum and/or Oryza and/or Avena with the aforementioned special physical characteristics in the antiperspirant cosmetic agents according to the invention results in targeted influencing of the sweat gland(s), although the invention is not limited to this theory. The targeted influencing of the sweat gland(s) can consist, for example, in the gellation of the at least one protein at pH values present exclusively within the excretory ducts of the sweat glands. In this way, effective blockage of the excretory ducts of the sweat glands can be ensured without reducing the antiperspirant effect of the cosmetic agent according to the invention through premature undesired gellation due to the addition of the at least one specific protein. Moreover, the targeted influencing of the sweat gland(s) can also consist, however, in the disruption of the load balance within the sweat gland(s), whereby the sweat production is influenced, particularly prevented. In this way, underarm perspiration is effectively reduced even in the absence of antiperspirant aluminum and/or zirconium halides and/or hydroxy halides.

According to the invention, the term “antiperspirant” is understood as the diminishing or reduction of the transpiration of the body's sweat glands.

Moreover, the term “aluminum and/or zirconium halides and/or hydroxy halides” is understood in relation to the present invention particularly as referring to aluminum and zirconium chlorides, bromides and iodides as well as compounds of the formulas Al(OH)_{y}X and Zr(OH)_{z}X, with X standing for a halide ion in the aforementioned formulas.

Furthermore, the term “cosmetic oil” is understood in terms of the present invention as being an oil that is suitable for cosmetic use that is not miscible with water in all quantities. The cosmetic oil used according to the invention is neither an odorant nor essential oil.

What is more, the term “odorant” is understood in terms of the present invention as referring to substances with a molar mass of 74 to 300 g/mol that include at least one osmophoric group in the molecule and have an odor and/or taste, that is, they are capable of stimulating the receptors of the hair cells of the olfactory system. Osmophoric groups are covalently bonded to the molecular skeleton in the form of hydroxy groups, formyl groups, oxo groups, alkoxycarbonyl groups, nitrile groups, nitro groups, azide groups, etc. In this connection, the term “odorant” also includes, in terms of the present invention, perfume oils, perfumes, or perfume oil components that are liquid at 20° C. and 1013 hPa.

Furthermore, the term “wax” is understood in the context of the present invention as a substance that is kneadable or solid to brittle and hard at 20° C., have a coarse to fine crystalline structure, and whose color is translucent to opaque but not glassy. Moreover, these substances melt above 25° C. without decomposing, are slightly fluid a little above the melting point (low viscosity), have a strongly temperature-dependent consistency and solubility and can be polished under slight pressure.

According to the invention, the term “protein” refers to chemical compounds which, through peptide bonds, form condensation products of amino acids that are linked in the manner of an acid amide. The number of amino acids in the proteins is preferably at least 2 and no more than 1000 amino acids. According to the invention, the term “protein” is to be understood as also including hydrolysates including protein fractions with different amino acid sequences and molecular weights. In relation to the present invention, this term is to be understood as also including mixtures of proteins occurring in Poaceae of the genus Triticum and/or Oryza and/or Avena.

Moreover, the term “sweet grass” is to be understood in the context of the present invention as referring to grass-like, anemophilous monocotyledonous plants. In terms of the present invention, Poaceae are particularly bamboos as well as the grain types of rye, barley, wheat, oats, corn, millet and rice.

Moreover, the expression “change in the light absorption of the at least one protein” is understood as referring both to the positive and negative change in the light transmission of the sample mixture, particularly of the protein solution, and to the absorption of light by the at least one protein or the sample mixture.

Furthermore, the term “change in pH” is understood as referring to a continuous change in the pH value. The continuous changing of the pH value can be achieved, for example, through the titration, or the uniform addition, of a base or acid.

According to the invention, the term “sample mixture” refers to a mixture of the at least one specific protein with a solvent, particularly water, buffer, or saline aqueous solutions.

In addition, the term “fatty acid” as it is used in relation to the present invention refers to aliphatic carboxylic acids having unbranched or branched carbon groups with 4 to 40 carbon atoms. The fatty acids used in relation to the present invention can be both naturally occurring and synthetically manufactured fatty acids. Moreover, the fatty acids can be mono- or polyunsaturated.

Finally, in relation to the present invention, the term “fatty alcohol” is to be understood as aliphatic, monovalent, primary alcohols having unbranched or branched hydrocarbon groups with 4 to 40 carbon atoms. The fatty alcohols used in the context of the invention can also be mono- or polyunsaturated.

Unless indicated otherwise, the expression “wt %” refers herein to the total weight of the cosmetic agents according to the invention.

The cosmetic agents according to the invention include, as the first component a), at least one substance selected from the group of cosmetic oils that are liquid at 20° C. and 1013 hPa, odorants and waxes. In the context of the present invention, the cosmetic oil that is liquid at 20° C. and 1013 hPa is selected from the group of (i) volatile cyclic silicone oils, particularly cyclic and linear silicone oils; (ii) volatile non-silicone oils, particularly liquid paraffin oils and isoparaffin oils; (iii) nonvolatile silicone oils; (iv) nonvolatile non-silicone oils; as well as (v) mixtures thereof.

According to the invention, the term “volatile oil” refers to oils which, at 20° C. and an ambient pressure of 1,013 hPa, have a vapor pressure from 2.66 to 40,000 Pa (0.02 to 300 mm Hg), preferably from 10 to 12,000 Pa (0.1 to 90 mm Hg), more preferably from 13 to 3,000 Pa (0.1 to 23 mm Hg), particularly from 15 to 500 Pa (0.1 to 4 mm Hg).

Moreover, in terms of the present invention, the term “nonvolatile oils” is understood as referring to oils which, at 20° C. and an ambient pressure of 013 hPa, have a vapor pressure of less than 2.66 Pa (0.02 mm Hg).

It can be preferred according to the invention to use mixtures of volatile silicone oils and volatile non-silicone oils in the antiperspirant cosmetic agents according to the invention, since this results in a drier feel on the skin. Moreover, it can be preferred in relation to the present invention if the antiperspirant cosmetic agents include a nonvolatile silicone oil and/or a nonvolatile non-silicone oil in order to mask insoluble components such as talcum or ingredients that have dried on the skin.

The use of mixtures of nonvolatile and volatile cosmetic oils is especially preferred according to the invention, since they enable parameters such as feel on the skin, visibility of the residue and stability of the antiperspirant cosmetic agent according to the invention to be adjusted, thereby better adapting the agent to consumers' needs.

The volatile and nonvolatile silicone oils as well as volatile and nonvolatile non-silicone oils that can be used in relation to the present invention are disclosed, for example, in published patent applications DE 102010063250 A1 and DE 102012222692 A1.

According to a preferred embodiment of the present invention, the cosmetic oil that is liquid at 20° C. and 1013 hPa is used in a total quantity of 0.02 to 98 wt %, preferably 2 to 85 wt %, more preferably 4 to 75 wt %, even more preferably 6 to 70 wt %, even more preferably 8 to 60 wt %, particularly 8 to 20 wt %, with respect to the total weight of the antiperspirant cosmetic agent.

At least one odorant can also be included as component a) of the cosmetic agents according to the invention. However, mixtures of different odorants are preferably used which, when combined, produce an appealing fragrance. Odorants that can be used in relation to the present invention are disclosed in DE 102010063250 A1, for example.

Especially pleasantly smelling antiperspirant cosmetic agents according to the invention are obtained if the at least one odorant is included in a total quantity of 0.00001 to 15 wt %, preferably 0.001 to 9 wt %, more preferably 0.01 to 8 wt %, even more preferably 0.1 to 7 wt %, even more preferably 0.2 to 6 wt %, particularly 0.2 to 2 wt %, with respect to the total weight of the antiperspirant cosmetic agent.

Moreover, the antiperspirant cosmetic agents according to the invention can include a wax as component a). This wax is preferably selected from the group of (i) fatty acid glycerin mono-, -di- and -triesters; (ii) Butyrospermum parkii (shea butter); (iii) esters of saturated, monovalent C₈₋₁₈ alcohols with saturated C₁₂₋₁₈ monocarboxylic acids; (iv) linear, primary C₁₂₋₂₄ alkanols; (v) esters from a saturated, monovalent C₁₆₋₆₀ alkanol and a saturated C₈₋₃₆ monocarboxylic acid; (vi) glycerin triesters of saturated linear C₁₂₋₃₀ carboxylic acids, which can be hydroxylated; (vii) natural plant waxes; (viii) animal waxes; (ix) synthetic waxes; as well as (x) mixtures thereof. Waxes that can be preferably used in relation to the present invention are disclosed in published patent application DE 102012222692 A1.

It is preferred in relation to the present invention if the wax is included in a total quantity of 0.01 to 50 wt %, preferably 3 to 40 wt %, more preferably 5 to 30 wt %, particularly 6 to 25 wt %, with respect to the total weight of the antiperspirant cosmetic agent.

According to one embodiment of the present invention, a provision can be made that the antiperspirant cosmetic agents according to the invention include, as component b), a propellant in a total quantity of 0 to 99 wt % with respect to the total weight of the antiperspirant cosmetic agent. If the cosmetic agents according to the invention include a propellant, it is preferably included in a total quantity of 1 to 98 wt %, preferably 20 to 90 wt %, more preferably 30 to 85 wt %, particularly 40 to 75 wt %, with respect to the total weight of the antiperspirant cosmetic agent. In this case, the cosmetic agents according to the invention are packaged as propellant gas-driven aerosols. Preferred propellants (propellant gases) are propane, propene, n-butane, iso-butane, iso-butene, n-pentane, pentene, iso-pentane, iso-pentene, methane, ethane, dimethylether, nitrogen, air, oxygen, nitrous oxide, 1,1,1,3-tetrafluorethane, heptafluoro-n-propane, perfluorethane, monochlorodifluoromethane, 1,1-difluorethane, and tetrafluoropropene, both individually and in mixtures. Hydrophilic propellant gases such as carbon dioxide, for example, can be used advantageously in terms of the present invention if the proportion of hydrophilic gases is selected so as to be low and lipophilic propellant gas (e.g., propane/butane) is present in excess. Propane, n-butane, iso-butane and mixtures of these propellant gases are especially preferred. It was found that the use of n-butane as the sole propellant gas can be especially preferred according to the invention.

As the third component c), the antiperspirant cosmetic agent according to the invention includes at least one specific protein from Poaceae of the genus Triticum and/or Oryza and/or Avena. The genus with the Latin name of Triticum designates Poaceae in the form of wheat and includes, for example, common wheat (also called soft wheat or bread wheat), spelt, as well as hard wheat (also called durum wheat). The Latin name of Oryza includes Poaceae in the form of rice, such as long-grain rice, short-grain rice, medium-grain rice, round-grain rice, and whole-grain rice. Finally, the Latin name of Avena refers to Poaceae in the form of oats.

An especially effective reduction of underarm perspiration through the at least one specific protein is achieved in the context of the present invention if the at least one protein is included in a total quantity of 0.5 to 60 wt %, preferably 1.0 to 50 wt %, more preferably 1.5 to 40 wt %, even more preferably 2.0 to 30 wt %, particularly 2.0 to 20 wt %, with respect to the total weight of the antiperspirant cosmetic agent. Without the intention of limiting the invention to this theory, the use of the abovementioned quantities of the at least one specific protein results in significant influencing of the sweat gland(s) through gellation of the protein in the excretory ducts of the sweat glands or by influencing the load balance within the sweat gland(s). An outstanding antiperspirant effect is ensured in this way. Moreover, the use of the abovementioned quantities of the at least one specific protein does not result in instable formulations, so the stability of the antiperspirant cosmetic agents according to the invention is ensured even over long periods of storage.

Especially good results are obtained with respect to the prevention and/or reduction of underarm wetness as well as to skin compatibility and storage stability if the at least one protein has an average molecular weight M_{w} of 150 to 100,000 Da, preferably 180 to 50,000 Da, more preferably 200 to 10,000 Da, even more preferably 250 to 8,000 Da, particularly 300 to 5,000 Da. The average molecular weight M_{w} can be determined through gel permeation chromatography (GPC), for example (Andrews P.; “Estimation of the Molecular Weights of Proteins by Sephadex Gel-Filtration”; Biochem. J., 1964, 91, pages 222 to 233). According to a preferred embodiment of the present invention, the at least one protein has an isoelectric point which lies in the range from pH 4.0 to pH 10.0, preferably pH 4.0 to pH 9.5, particularly pH 4.0 to pH 8.0. Particularly proteins having an isoelectric point in the abovementioned pH range have proven to be advantageous in relation to the present invention with regard to the antiperspirant effect and the stability of the cosmetic agents according to the invention.

An especially good antiperspirant effect, skin compatibility and storage stability is ensured in the context of the present invention if the at least one protein brings about a change in the light absorption upon a change in pH of at least 0.5 in a pH range from pH 4.5 to pH 7.5, particularly pH 5.0 to pH 7.0, with a concentration of 0.001 to 10 wt % protein, with respect to the total weight of the sample mixture used for the pH measurement, and a temperature of 20° C. Without the intention of limiting the invention to this theory, the use of the at least one specific protein that brings about a change in the light absorption in a certain pH range results in a significantly increased influencing of the sweat gland(s) through pH-selective gellation in the excretory ducts of the sweat glands or through disruption of the load balance of the sweat gland(s), thereby ensuring an outstanding antiperspirant effect of the cosmetic agents according to the invention that is comparable to the antiperspirant effect of aluminum salt-including or aluminum-zirconium salt-including cosmetic agents of the prior art.

In relation to the present invention, it is preferred if the change in pH is achieved through the addition of hydrogen carbonates or carbonates, particularly of sodium hydrogen carbonates.

According to a preferred embodiment of the present invention, the at least one protein is selected from the group of (i) non-modified proteins; (ii) hydrolyzed proteins; (iii) chemically modified proteins, particularly hydrophobically and/or cationically and/or anionically modified proteins; (iv) physically modified proteins, particularly fractionated and/or purified and/or irradiated proteins; (v) hydrolyzed non-modified proteins; (vi) hydrolyzed and chemically modified proteins, particularly hydrolyzed and hydrophobically and/or cationically and/or anionically modified proteins; (vii) hydrolyzed and physically modified proteins, particularly fractionated and/or purified and/or irradiated proteins; as well as (viii) mixtures thereof.

According to the invention, the term “non-modified protein” is to be understood as referring to proteins that have been treated neither by means of chemical processes, such as hydrolysis or chemical modification, for example, nor by means of physical methods, such as purification, separation and irradiation, for example.

Furthermore, the terms “hydrolyzed protein” and “protein hydrolysate” are to be understood according to the invention as proteins that were prepared through chemical, particularly alkaline or acidic hydrolysis, through enzymatic hydrolysis and/or through a combination of these two types of hydrolysis. All hydrolytically active enzymes, such as alkaline proteases, for example, are suitable for the enzymatic decomposition. Overviews of the preparation of protein hydrolysates have been published by G. Schuster and A. Domsch in Seifen Öle Fette Wachse 108, (1982) 177 and Cosm.Toil. 99, (1984) 63, by H. W. Steisslinger in ParfKosm. 72, (1991) 556, and F. Aurich et al. in Tens.SurfDet. 29, (1992) 389. According to the invention, mixtures of individual amino acids that are obtained merely by mixing the pure substances of the amino acids, as well as total hydrolysates that consist only of individual amino acids do not fall under the terms “hydrolyzed protein” and “protein hydrolysate.”

Moreover, the term “chemically modified protein” is to be understood in relation to the present invention as referring to proteins that are obtained through chemical conversion of the reactive groups of the proteins, particularly of the hydroxy, amine, imidazole, guanidine and/or thiol groups of the side chains of the amino acids of the protein, with hydrophobic and/or cationic and/or anionic compounds.

In addition, the term “physically modified protein” is to be understood in terms of the present invention as referring to proteins that have been modified through physical intervention, particularly through heat and/or light and/or fractionation.

In the context of this embodiment, it is especially preferred if the at least one protein is selected from the group of chemically modified, particularly hydrophobically modified, proteins. In this connection, the hydrophobically modified protein has one or more C₄₋₃₀ carbon chains, it being possible for the C₄₋₃₀ hydrocarbon chains to be linear, cyclic, branched, unbranched, saturated, unsaturated and aromatic, and the C₄₋₃₀ hydrocarbon chains being bonded to the protein residue via ether and/or ester and/or amine and/or amide bonds.

Moreover, it is preferred in the context of this embodiment if the at least one protein is selected from the group of chemically modified, particularly cationically modified, proteins. Preferably, the cationically modified protein therefore includes one or more residue(s) of the formula R—N⁺(CH₃)₂—CH₂—CH(OH)—CH₂—X—R, in which R stands for an alkyl group with 1 to 30 carbon atoms, an alkenyl group with 1 to 30 carbon atoms, a hydroxyalkyl group with 1 to 30 carbon atoms, particularly for a methyl group, a C₁₀₋₁₈ alkyl or a C₁₀₋₁₈ alkenyl group, X stands for O, N or S, and R stands for the protein residue. The cationizing of the proteins with the residues described above can be achieved by converting the proteins with the corresponding halides of the above formula, it being possible for the previously described residues to be bonded to the protein via ether and/or ester and/or amide and/or amine bonds. In relation to the present invention, the term “protein residue” is to be understood as being the backbone of the corresponding protein formed by the linking of amino acids, to which protein the cationic group is bonded via the abovementioned bonds.

According to another preferred embodiment of the present invention, the at least one protein occurs in Poaceae of the genus Triticum (wheat).

In this connection, it can be preferred if the at least one protein is a cationically functionalized and/or hydrolyzed protein from Poaceae of the genus Triticum (wheat).

Furthermore, it is especially preferred in the context of this embodiment if the at least one protein includes one or more residue(s) of the formula R—N⁺(CH₃)₂—CH₂—CH(OH)—CH₂—X—R, in which R stands for a methyl group, a C₁₀₋₁₈ alkyl or a C₁₀₋₁₈ alkenyl group, X stands for O, N or S, and R stands for the optionally hydrolyzed protein residue.

With respect to the antiperspirant effect, skin compatibility and storage stability of the cosmetic agents according to the invention, it is especially preferred if the at least one protein is a protein isolated from Poaceae of the genus Triticum (wheat), with the protein being hydrolyzed and/or with the protein being functionalized with at least one cationic group selected from lauryldimonium hydroxypropyl groups, hydroxypropyltrimonium groups, as well as mixtures thereof. Without the intention of limiting the invention to this theory, the use of these specific proteins results in a significantly increased influencing of the sweat gland(s) through pH-selective gellation or disruption of the load balance within the excretory ducts of the sweat glands. In this way, an outstanding antiperspirant effect of the cosmetic agents according to the invention is ensured which is comparable to the antiperspirant effect of aluminum salt-containing or aluminum-zirconium salt-containing cosmetic agents of the prior art. Moreover, the use of these specific proteins does not result in a negative interaction with other ingredients in the antiperspirant cosmetic agent, thereby ensuring the good storage stability of the antiperspirant cosmetic agents according to the invention. What is more, the cosmetic agents according to the invention have good skin compatibility. It has proven advantageous in this connection if the at least one protein includes lysine in 0 to 1.5 mol %, histidine in 0.7 to 2.0 mol % and arginine in 2.0 to 3.0 mol % with respect to the total substance quantity of all amino acids of the protein. The substance proportion of the abovementioned amino acids can be determined chromatographically, for example, using sulfonated polystyrene resins (Moore S. et al.; “Chromatography of amino acids on sulfonated polystyrene resin”; J. of Biological Chem., 1951, 192, pages 663 to 681).

Moreover, it has proven advantageous in this connection if the at least one protein has a calcium content of 4 to 400 mg, a potassium content of 50 to 800 mg, a phosphorous content of 1600 to 3200 mg, and a sulfur content of 1300 to 1700 mg, each with respect to 1 kg of the at least one protein. The contents indicated above can be determined, for example, by means of atom emission spectrometry (ICP-OES) after microwave digestion with nitric acid (A. Oliveira et al.: “Evaluation of Metal Ions in Rice Samples: Extraction and Direct Determination by ICP-OES,” J. Braz. Chem. Soc, 2012, 23, pages 838 to 845).

According to another especially preferred embodiment of the present invention, the at least one protein brings about a change in light absorption of 1.5 to 90%, preferably 2 to 80%, more preferably 2.5 to 70%, even more preferably 3 to 65%, particularly 3.5 to 60%. Particularly proteins from Poaceae of the genus Triticum and/or Oryza and/or Avena that bring about the abovementioned change in light absorption result in an outstanding antiperspirant effect in the context of the present invention. The change in light absorption can occur through a change in the light transmission of the sample mixture, particularly through clouding, and through the absorption of light by the sample mixture, particularly by the protein itself.

The changes in light absorption upon a change in pH of at least 0.5 on which the present invention is based can be determined by measuring the light transmission of a light beam through the sample mixture. The measurements of the light transmission are performed using a Metrohm 6.1115.000 optrode at a wavelength of 574 nm (greenish yellow) in mV (resolution 0.1 mV) in an open sample vessel at 23° C. and 1013 mbar. The change in pH in the pH range from 4.0 to 8.0 is achieved through the slow and continuous addition of a carbonate or hydrogen carbonate solution, preferably of a 1 wt % sodium hydrogen carbonate solution, to a sample mixture under measurement of the pH value using a pH electrode and under stirring at a speed of 750 to 850 rpm. The change in light absorption that is brought about by the at least one protein is calculated according to the formula ΔL=[(|Lᵢ|/|L₀|]*100. In this formula, Lᵢ stands for the after the change in the pH by at least 0.5 in the pH range from 4.0 to 8.0, preferably pH 4.5 to 7.5, particularly pH 5.0 to 7.0. In this formula, L₀ stands for the difference of the light transmissions at pH 4.0 and at pH 8.0, preferably at pH 4.5 and at pH 7.5, particularly at pH 5.0 and at pH 7.0—that is, the light transmission at pH 8.0 minus the light transmission at pH 4.0, for example. The at least one specific protein in the antiperspirant cosmetic agents according to the invention brings about a change in the light absorption of 1 to 100% determined according to the above method. However, the present invention is not limited to antiperspirant cosmetic compositions including at least one specific protein that brings about a change in the light absorption of 1 to 100% determined according to the above method. It also includes antiperspirant cosmetic compositions including at least one specific protein that brings about a change in the light absorption of 1 to 100% determined according to other methods.

In relation to the present invention, it is preferred if the concentration of the at least one protein in the mixture used to determine the change in light absorption is from 0.005 to 10 wt %, preferably 0.05 to 5 wt %, more preferably 0.07 to 3 wt %, particularly 0.09 to 2 wt %, with respect to the total weight of the sample mixture used to determine the change in light absorption.

According to the invention, the at least one protein preferably brings about a change in light absorption upon a change in pH of at 0.5 and no more than 3.5, preferably of at least 0.5 and no more than 2.5, particularly of at least 0.5 and no more than 1.5. The change in pH can be achieved in the appropriate amount particularly through the addition of acids or bases, preferably bases in the form of carbonates or hydrogen carbonates.

According to another preferred embodiment of the present invention, the antiperspirant cosmetic agent has a pH of 2 to 10. Within this range, a stable formulation of the cosmetic agents according to the invention is possible without the occurrence of undesired interactions between the ingredients of the antiperspirant cosmetic agents according to the invention. The setting of the desired pH value can be achieved according to the invention through the use of acids and bases that are known to a person skilled in the art and common in antiperspirant cosmetic agents.

According to the invention, it is also preferred if the antiperspirant cosmetic agent additionally includes at least one preservative. Preservatives that are preferred according to the invention are formaldehyde cleavers iodopropynyl butylcarbamate, parabens, phenoxyethanol, ethanol, benzoic acid and salts thereof, dibromodicyanobutane, 2-bromo-2-nitro-propane-1,3-diol, imidazolidinyl urea, 5-chloro-2-methyl-4-isothiazolin-3-one, 2-chloroacetamide, benzalkonium chloride, benzyl alcohol, salicylic acid and salicylates. Other preservatives that can be used in the context of the present invention are the substances listed in Annex 6 of the Cosmetics Ordinance as well as cosmetic raw materials with preservative characteristics or raw materials that support or potentiate the preservative effect of the aforementioned preservatives. The preservatives are preferably included in a total quantity of 0.01 to 10 wt %, preferably 0.1 to 7 wt %, more preferably 0.2 to 5 wt %, particularly 0.3 to 2.0 wt % with respect to the total weight of the antiperspirant cosmetic agent.

In relation to the present invention, it is preferred if the antiperspirant cosmetic agent is present as a water-in-oil emulsion. Particularly, this can be a sprayable water-in-oil emulsion that can be sprayed by means of a propellant. In this connection, it is preferred if the antiperspirant cosmetic agent according to the invention present in the form of a water-in-oil emulsion includes the at least one protein in a total quantity of 0.1 to 70 wt %, preferably 0.5 to 60 wt %, more preferably 1.0 to 50 wt %, even more preferably 1.5 to 40 wt %, even more preferably 2.0 to 30 wt %, particularly 2.0 to 20 wt %, with respect to the total weight of the antiperspirant cosmetic agent.

It can be equally preferred according to the invention, however, if the antiperspirant cosmetic agent is present as an oil-in-water emulsion. In this case, the cosmetic agent according to the invention is preferably applied as a propellant-free pump spray or squeeze spray or as a roll-on. In this connection, it is preferred if the antiperspirant cosmetic agent present in the form of a oil-in-water emulsion includes the at least one protein in a total quantity of 0.1 to 70 wt %, preferably 0.5 to 60 wt %, more preferably 1.0 to 50 wt %, even more preferably 1.5 to 40 wt %, even more preferably 2.0 to 30 wt %, particularly 2.0 to 20 wt %, with respect to the total weight of the antiperspirant cosmetic agent.

According to another preferred embodiment of the present invention, the cosmetic agents according to the invention can include only a small amount of free water or no free water. In terms of the present invention, free water is understood as water that is different from water of crystallization, hydration water, or similar molecularly bound water of the components used. The antiperspirant cosmetic agent preferably includes free water in a total quantity of less than 10 wt %, preferably less than 8 wt %, more preferably less than 5 wt %, even more preferably less than 3 wt %, even more preferably less than 1 wt %, particularly 0 wt %, with respect to the total weight of the antiperspirant cosmetic agent.

In the context of another embodiment, however, it is also preferred if the antiperspirant cosmetic agent is present as an aqueous, aqueous-alcoholic or aqueous-glycolic solution. Since the cosmetic agents according to the invention do not include any antiperspirant aluminum and/or zirconium halides and/or hydroxy halides that have a reduced antiperspirant effect as a result of the addition of protic solvents, protic solvents such as aqueous solutions can be used according to the invention to formulate the antiperspirant cosmetic agents according to the invention without a significant reduction in the antiperspirant effect. Therefore, even when protic solvents are used, the use of the at least one specific protein ensures effective influencing of the sweat gland(s) and hence an outstanding antiperspirant effect.

In connection with this embodiment of the present invention, it was surprisingly found that the influencing of the sweat gland(s) through the at least one specific protein can be increased significantly if the antiperspirant cosmetic agents according to the invention include free water in a quantity of 5 to 99 wt % with respect to the total weight of the antiperspirant cosmetic agent. In an especially preferred embodiment of the present invention, the antiperspirant cosmetic agent therefore includes free water in a total quantity of 5 to 96 wt %, preferably 15 to 80 wt %, more preferably 30 to 70 wt %, particularly 40 to 60 wt %, with respect to the total weight of the antiperspirant cosmetic agent.

Furthermore, it is preferred in connection with this embodiment if the antiperspirant cosmetic agent includes ethanol in a total quantity of 1 to 99 wt %, preferably 5 to 70 wt %, more preferably 7 to 50 wt %, particularly 10 to 30 wt %, with respect to the total weight of the antiperspirant cosmetic agent. As stated above, through the use of the at least one specific protein, even high quantities of protic solvents such as ethanol can be used without negatively impacting the antiperspirant effect of the antiperspirant cosmetic agent according to the invention.

The application of the antiperspirant cosmetic agent according to the invention can be achieved using different methods. According to a preferred embodiment, the antiperspirant cosmetic agent is packaged as a spray application. The spray application is performed using a spraying device including a charge of the liquid, viscous/flowable, suspension-like or powdered antiperspirant cosmetic agent according to the invention in a container. The charge can be under the pressure of a propellant (pressurized gas cans, pressurized gas packaging, aerosol packaging), or a mechanically operated pump atomizer without propellant gas (pump sprays/squeezy bottle). The atomization of the antiperspirant cosmetic agent can be achieved physically, mechanically, or electromechanically, for example through piezo effects or electric pumps. Containers and removal devices than can be used in the context of this embodiment are described in published patent application DE 102012222692 A1, for example.

The antiperspirant cosmetic agent can also be preferably packaged as a stick, soft solid, gel, roll-on, or as a loose or compact powder. The formulation of the antiperspirant cosmetic agents according to the invention in a specific dosage form, such as a roll-on antiperspirant, antiperspirant stick or an antiperspirant gel, is preferably oriented around the requirements of the intended use. Depending on the intended use, the antiperspirant cosmetic agents according to the invention can therefore be present in solid, semisolid, liquid, dispersed, emulsified, suspended, gel-like, multiphase or powdered form. In terms of the present invention, the term “liquid” includes any type of solid dispersions in liquids. Moreover, in terms of the present invention, multiphase antiperspirant cosmetic agents according to the invention are understood as being agents having at least 2 different phases with a phase separation and in which the phases can be arranged horizontally, that is, over one another, or vertically, that is, next to one another. Application can be achieved using a roll-on applicator or a solid stick, for example.

It can also be preferred in relation to the present invention if the antiperspirant cosmetic agent is included on and/or in a disposable substrate, selected from the group of cloths, pads and wads. Wet wipes—that is, moist towelettes that are prefabricated for the used and preferably packaged individually, such as those which are well known from the area of glass cleaning or from the area of moist toilet papers—are especially preferred. Such wet wipes, which can advantageously also include preservatives, are impregnated or loaded with an antiperspirant cosmetic agent according to the invention and preferably packaged individually. Preferred substrate materials are selected from porous flat cloths. These wipes include cloths made of woven and nonwoven (fleece), synthetic and natural fibers, felt, paper or foam, such as hydrophilic polyurethane foam. Deodorizing or antiperspirant substrates that are preferred according to the invention can be obtained by soaking or impregnation, or even by melting an antiperspirant cosmetic agent according to the invention onto a substrate.

It is preferred according to the invention if the antiperspirant cosmetic agent includes at least one adjuvant selected from the group of (i) emulsifiers and/or surfactants; (ii) thickeners; (iii) chelating agents; (iv) deodorant agents; (v) mono- and/or polyvalent alcohols and/or polyethylene glycols; (vi) skin-cooling agents; (vii) pH adjusters; (viii) skin care agents such as moisturizers, skin-soothing agents, skin-clarifying agents, skin-smoothing agents; as well as (ix) mixtures thereof.

Emulsifiers and surfactants that are preferably suitable according to the invention are selected from anionic, cationic, nonionic, amphoteric, particularly ampholytic and zwitterionic emulsifiers and surfactants. Surfactants are amphiphilic (bifunctional) compounds that consists of at least one hydrophobic and at least one hydrophilic moiety. The hydrophobic group is preferably a hydrocarbon chain with 8 to 28 carbon atoms, which can be saturated or unsaturated, linear or branched. Especially preferably, this C₈-C₂₈ alkyl chain is linear. Emulsifiers and surfactants that can be preferably used in relation to the present invention are disclosed in published patent applications DE 102012222692 A1, DE 102010063250 A1, and DE 102010055816 A1, for example.

To thicken the antiperspirant cosmetic agents according to the invention, substances are preferably used which are selected from cellulose ethers, xanthan gum, sclerotium gum, succinoglycans, polygalactomannans, pectins, agar, carragheen (carrageenan), traganth, gum arabic, karaya gum, tara gum, gellan, gelatins, propylene glycol alginate, alginic acids and salts thereof, polyvinyl pyrrolidones, polyvinyl alcohols, polyacrylamides, physically modified (e.g., through pregelatinization) and salts thereof chemically modified starches, acrylic acid acrylate copolymers, acrylic acid acrylamide copolymers, acrylic acid vinyl pyrrolidone copolymers, acrylic acid vinyl formamide copolymers, and polyacrylates. Especially preferred thickeners are also selected from among the carbomers. Carbomers are thickening crosslinked polymers of acrylic acid, methacrylic acid, and salts thereof. The crosslinking can be achieved by means of polyfunctional compounds, such as polyalkylene ethers of polysaccharides or polyalcohols, for example sucrose allyl ether, pentaerythritol allyl ether, propylene allyl ether. Homopolymers of acrylic acid or salts thereof that are crosslinked with a pentaerythritol allyl ether, a sucrose allyl ether or a propylene allyl ether are preferred in relation to the present invention. A thickener that can be used in the context of the present invention is a copolymer of C₁₀₋₃₀ alkyl acrylate, acrylic acid, methacrylic acid, as well as esters thereof, that is crosslinked with a sucrose allyl ether or a pentaerythritol allyl ether. Carbomer-based thickeners are the products available under the trade names Carbopol® (BF Goodrich, Ohio, USA), such as Carbopol 934, Carbopol 940, Carbopol 941, Carbopol 971, Carbopol 974, Carbopol EZ2, Carbopol ETD 2001, Carbopol ETD 2020, Carbopol ETD 2050, Carbopol ultrez 10, Carbopol ultrez 20, or Carbopol ultrez 21, for example.

Moreover, lipophilic thickeners can be used to thicken the antiperspirant cosmetic agents according to the invention. Lipophilic thickeners which are especially preferred according to the invention are selected from hydrophobized clay materials, bentonites, pyrogenic silicic acids, and derivatives thereof.

To further support the influencing of the sweat gland(s) through the at least one specific protein, it can be advantageous to add at least one chelating agent to the antiperspirant cosmetic agents according to the invention in a total quantity of 0.01 to 3.0 wt %, preferably 0.02 to 1.0 wt %, particularly 0.05 to 0.1 wt %, with respect to the total weight of the antiperspirant agent according to the invention. In relation to the present invention, preferred chelating agents are selected from the group of β-alanine diacetic acid, cyclodextrin, diethylene triamine pentamethylene phosphonic acid, sodium, potassium, calcium disodium, ammonium and triethanolamine salts of ethylenediaminetetraacetic acid (EDTA), etidronic acid, hydroxyethylethylenediaminetetraacetic acid (HEDTA) and sodium salts thereof, sodium salts of nitrilotriacetic acid (NTA), diethylenetriaminepentaacetic acid, phytic acid, hydroxypropyl cyclodextrin, methyl cyclodextrin, pentasodium aminotrimethylene phosphonate, pentasodium ethylenediamine tetramethylene phosphonate, pentasodium diethylenetriamine pentaacetate, pentasodium triphosphate, potassium EDTMP, sodium EDTMP, sodium dihydroxyethyl glycinate, sodium phytate, sodium polydimethylglycinophenol sulfonate, tetrahydroxyethyl ethylenediamine, tetrahydroxypropyl ethylenediamine, tetrapotassium etidronate, tetrasodium etidronate, tetrasodium iminodisuccinate, trisodium ethylenediamine disuccinate, tetrasodium-N,N-bis(carboxymethyl) glutamate, tetrasodium-DL-alanine-N,N-diacetate and desferrioxamine.

The deodorizing effect of the antiperspirant cosmetic agents according to the invention can be further increased if at least one deodorizing agent with an antibacterial and/or bacteriostatic and/or enzyme-inhibiting and/or odor-neutralizing and/or odor-absorbing effect is included in a total quantity of 0.0001 to 40 wt %, preferably 0.2 to 20 wt %, more preferably 1 to 15 wt %, particularly 1.5 to 5 wt %, with respect to the total weight of the antiperspirant cosmetic agent according to the invention. Insofar as ethanol is used in the agents according to the invention, it is not regarded in the context of the present invention as being a deodorant agent, but rather as a component of the vehicle. Deodorant agents that are preferred according to the invention are disclosed in published patent application DE 102010063250 A1, for example. Preferred antiperspirant cosmetic agents according to the invention also include at least one water-soluble polyvalent C₂₋₉ alkanol with 2 to 6 hydroxyl groups and/or at least one water-soluble polyethylene glycol with 3 to 50 ethylene oxide units as well as mixtures thereof. These do not include the abovementioned deodorant agents in the form of 1,2-alkanediols. Preferred alkanols and water-soluble polyethylene glycols are described in published patent application DE 102010063250 A1, for example.

According to another embodiment of the present invention, the antiperspirant cosmetic agents also include at least one skin-cooling agent. Examples of skin-cooling agents which are suitable according to the invention are menthol, isopulegol and menthol derivatives, e.g., menthyl lactate, menthyl glycolate, menthyl ethyl oxamate, menthyl pyrrolidone carboxylic acid, menthyl methyl ether, menthoxy propanediol, menthone glycerin acetal (9-methyl-6-(1-methylethyl)-1,4-dioxaspiro (4. 5)decan-2-methanol), monomenthyl succinate, 2-hydroxymethyl-3,5,5-trimethylcyclohexanol and 5-methyl-2-(1-methylethyl)cyclohexyl-N-ethyl oxamate. Preferred skin-cooling agents are menthol, isopulegol, menthyl lactate, menthoxy propanediol, menthylpyrrolidone carboxylic acid and 5-methyl-2-(1-methylethyl)cyclohexyl-N-ethyl oxamate, as well as mixtures of these substances, particularly mixtures of menthol and menthyl lactate, menthol, menthol glycolate and menthyl lactate, menthol and menthoxy propanediol or menthol and isopulegol.

Acids and/or alkalizing agents and/or buffers are preferred according to the invention for use as pH adjusters. According to the invention, inorganic acids (such as hydrochloric acid, sulfuric acid or phosphoric acid, for example) or organic acids (such as citric acid, tartaric acid or malic acid, for example) are preferred according to the invention as acids. The alkalizing agents that can be used according to the invention are preferably selected from the group consisting of ammoniac, basic amino acids, alkali hydroxides, carbonates and hydrogen carbonates, alkanolamines, for example amino-2-methyl-1-propanol, monoethanolamine, triethanolamine, diethanolamine, and triisopropanolamine, alkaline metal metasilicates, urea, morpholine, N-methylglucamine, imidazole, alkali phosphates and alkali hydrogen phosphates. Lithium, sodium, or potassium, and particularly sodium or potassium, are preferably used as alkali metal ions. Buffer systems that are suitable in relation to the present invention are, in particular, carbonic acid bicarbonate buffers, carbonic acid silicate buffers, acetic acid acetate buffers, phosphate buffers, ammoniac buffers, citric acid or citrate buffers, buffers based on tris(hydroxymethyl) aminomethane, buffers based on 4-(2-hydroxyethyl)-1-piperazine ethane sulfonic acid, buffers based on 4-(2-hydroxyethyl)-piperazine-1-propanesulfonic acid, buffers based on 2-(N-morpholino)ethanesulfonic acid, as well as barbital acetate buffers. The selection of the corresponding buffer system is oriented here around the desired pH value of the antiperspirant cosmetic agents according to the invention. In a preferred embodiment, the antiperspirant cosmetic agents according to the invention are characterized in that they include, with respect to the total weight of the antiperspirant cosmetic agent according to the invention,
at least one protein in a total quantity of 0.5 to 60 wt %, preferably 1.0 to 50 wt %, more preferably 1.5 to 40 wt %, even more preferably 2.0 to 30 wt %, particularly 2.0 to 20 wt %,
12 to 98 wt %, preferably 25 to 55 wt %, more preferably 30 to 50 wt %, particularly 35 to 45 wt % water,
at least one emulsifier and/or surfactant,
at least one pH adjuster,
at least one preservative, and
at least one substance selected from the group of cosmetic oils that are liquid at 20° C. and 1013 hPa, odorants and waxes,
   with the at least one protein occurring in Poaceae of the genus Triticum and/or Oryza and/or Avena and with the at least one protein bringing about a change in the light absorption of 1 to 100% when the pH changes from at least 0.5 to a pH range of 4.0 to pH 8.0, a temperature of 20° C. to 40° C. and a concentration of the protein of 0.001 to 10 wt %, with respect to the total weight of the sample mixture used to determine the change in light absorption.

In another preferred embodiment, the antiperspirant cosmetic agents according to the invention are characterized in that they include, with respect to the total weight of the antiperspirant cosmetic agent according to the invention,
at least one protein in a total quantity of 0.5 to 60 wt %, preferably 1.0 to 50 wt %, more preferably 1.5 to 40 wt %, even more preferably 2.0 to 30 wt %, particularly 2.0 to 20 wt %,
12 to 98 wt %, preferably 25 to 55 wt %, more preferably 30 to 50 wt %, particularly 35 to 45 wt % water,
at least one emulsifier and/or surfactant,
at least one pH adjuster,
at least one preservative,
0.01 to 2 wt %, preferably 0.1 to 1 wt %, more preferably 0.2 to 0.7 wt %, particularly 0.3 to 0.5 wt % of a thickener, and
at least one substance selected from the group of cosmetic oils that are liquid at 20° C. and 1013 hPa, odorants and waxes,
   with the at least one protein occurring in Poaceae of the genus Triticum and/or Oryza and/or Avena and with the at least one protein bringing about a change in the light absorption of 1 to 100% when the pH changes from at least 0.5 to a pH range of 4.0 to pH 8.0, a temperature of 20° C. to 40° C. and a concentration of the protein of 0.001 to 10 wt %, with respect to the total weight of the sample mixture used to determine the change in light ab sorption.

In a preferred embodiment, the antiperspirant cosmetic agents according to the invention are characterized in that they include, with respect to the total weight of the antiperspirant cosmetic agent according to the invention,
at least one protein in a total quantity of 0.5 to 60 wt %, preferably 1.0 to 50 wt %, more preferably 1.5 to 40 wt %, even more preferably 2.0 to 30 wt %, particularly 2.0 to 20 wt %,
12 to 98 wt %, preferably 25 to 55 wt %, more preferably 30 to 50 wt %, particularly 35 to 45 wt % water,
at least one propellant in a total quantity of 1 to 98 wt %, preferably 20 to 90 wt %, more preferably 30 to 85 wt %, particularly 40 to 75 wt %,
at least one emulsifier and/or surfactant,
at least one pH adjuster,
at least one preservative, and
at least one substance selected from the group of cosmetic oils that are liquid at 20° C. and 1013 hPa, odorants and waxes,
   with the at least one protein occurring in Poaceae of the genus Triticum and/or Oryza and/or Avena and with the at least one protein bringing about a change in the light absorption of 1 to 100% when the pH changes from at least 0.5 to a pH range of 4.0 to pH 8.0, a temperature of 20° C. to 40° C. and a concentration of the protein of 0.001 to 10 wt %, with respect to the total weight of the sample mixture used to determine the change in light ab sorption.

In another preferred embodiment, the antiperspirant cosmetic agents according to the invention are characterized in that they include, with respect to the total weight of the antiperspirant cosmetic agent according to the invention,
at least one protein in a total quantity of 0.5 to 60 wt %, preferably 1.0 to 50 wt %, more preferably 1.5 to 40 wt %, even more preferably 2.0 to 30 wt %, particularly 2.0 to 20 wt %,
12 to 98 wt %, preferably 25 to 55 wt %, more preferably 30 to 50 wt %, particularly 35 to 45 wt % water,
at least one propellant in a total quantity of 1 to 98 wt %, preferably 20 to 90 wt %, more preferably 30 to 85 wt %, particularly 40 to 75 wt %,
at least one emulsifier and/or surfactant,
at least one pH adjuster,
at least one preservative,
0.01 to 2 wt %, preferably 0.1 to 1 wt %, more preferably 0.2 to 0.7 wt %, particularly 0.3 to 0.5 wt % of a thickener, and
at least one substance selected from the group of cosmetic oils that are liquid at 20° C. and 1013 hPa, odorants and waxes,
   with the at least one protein occurring in Poaceae of the genus Triticum and/or Oryza and/or Avena and with the at least one protein bringing about a change in the light absorption of 1 to 100% when the pH changes from at least 0.5 to a pH range of 4.0 to pH 8.0, a temperature of 20° C. to 40° C. and a concentration of the protein of 0.001 to 10 wt %, with respect to the total weight of the sample mixture used to determine the change in light ab sorption.

In relation to the present invention, a provision can also be made that the cosmetic agent according to the invention is packaged as a two-component agent. To this end, the individual components are stored in separate containers and applied successively in any sequence or simultaneously onto the skin. The separation into multi-component systems is preferred particularly in cases in which incompatibilities among the ingredients can be expected or are feared.

Another object of the present invention is therefore a packaging unit (kit of parts) which comprises, packaged separately from one another,
a) at least one first container (C1), including a cosmetic agent (M1) comprising at least one antiperspirant agent, and
b) at least one second container (C2), including a cosmetic agent (M2) comprising at least one protein, with the at least one protein occurring in Poaceae of the genus Triticum and/or Oryza and/or Avena, with the at least one protein bringing about a change in the light absorption of 1 to 100% when the pH changes from at least 0.5 to a pH range of 4.0 to pH 8.0, a temperature of 20° C. to 40° C. and a concentration of the protein of 0.001 to 10 wt %, with respect to the total weight of the sample mixture used to determine the change in light absorption, and with the cosmetic agent including no aluminum and/or zirconium halides and/or hydroxy halides.

According to the invention, the term “antiperspirant agent” is understood as referring to agents that prevent or reduce the perspiration of the sweat glands of the body. However, the term does not include the proteins Poaceae of the genus Triticum and/or Oryza and/or Avena included in the cosmetic agent (M2) that bring about a change in light absorption under the previously described conditions. As regards the cosmetic agent (M2) in the container (C2), the remarks made concerning the cosmetic agents according to the invention apply mutatis mutandis.

Another object of the present invention is the use of a protein for the at least partial influencing of the sweat gland(s), with the at least one protein occurring in Poaceae of the genus Triticum and/or Oryza and/or Avena, and with the at least one protein bringing about a change in the light absorption of 1 to 100% when the pH changes from at least 0.5 to a pH range of pH 4.0 to pH 8.0, a temperature of 20° C. to 40° C., and a concentration of the protein of 0.001 to 10 wt %, with respect to the total weight of the sample mixture used to determine the change in light absorption.

According to the invention, the influencing of the sweat gland(s) is understood to the effect that the excretion of sweat from the excretory duct is prevented or reduced. Without the intention of limiting the invention to this theory, this can occur through the formation of a gel and/or precipitation of the at least one specific protein in the excretory duct of the sweat gland or the excretory ducts of the sweat glands. Moreover, the use of the at least one specific protein can also result in a disruption of the load balance within the excretory ducts of the sweat glands, however. As regards the use according to the invention, the remarks concerning the antiperspirant cosmetic agents according to the invention according to the invention apply mutatis mutandis.

Moreover, another object of the present invention is the use of a combination including
a) at least one substance selected from the group of cosmetic oils that are liquid at 20° C. and 1013 hPa, odorants and waxes,
b) propellant in a total quantity of 0 to 99 wt % with respect to the total weight of the antiperspirant cosmetic agent, and
c) at least one protein in a total quantity of 0.1 to 70 wt % with respect to the total weight of the antiperspirant cosmetic agent, with the at least one protein occurring in Poaceae of the genus Triticum and/or Oryza and/or Avena, and with the at least one protein bringing about a change in the light absorption of 1 to 100% when the pH changes from at least 0.5 to a pH range of 4.0 to pH 8.0, a temperature of 20° C. to 40° C. and a concentration of the protein of 0.001 to 10 wt %, with respect to the total weight of the sample mixture used to determine the change in light absorption, and
   with the combination including no aluminum and/or zirconium halides and/or hydroxy halides for reducing and/or preventing sweat, particularly underarm perspiration or sweat in other areas of the body.

In terms of the present invention, the term “combination” includes a mixture of the abovementioned ingredients a), b), and c). As regards the use of the abovementioned combination, the remarks made concerning the antiperspirant cosmetic agents according to the invention and the use according to the invention apply mutatis mutandis.

In addition, another object of the present invention is an antiperspirant cosmetic agent including
a) at least one substance selected from the group of cosmetic oils that are liquid at 20° C. and 1013 hPa, odorants and waxes,
b) propellant in a total quantity of 0 to 99 wt % with respect to the total weight of the antiperspirant cosmetic agent, and
c) at least one protein isolated from Poaceae of the genus Triticum (wheat) in a total quantity of 0.1 to 70 wt % with respect to the total weight of the antiperspirant cosmetic agent, with the protein being hydrolyzed and/or with the protein being functionalized with at least one cationic group selected from lauryldimonium hydroxypropyl groups, hydroxypropyltrimonium groups and mixtures thereof,
   with the antiperspirant cosmetic agent not including any aluminum and/or zirconium halides and/or hydroxy halides.

In the context of this object, it is particularly preferred if the at least one protein includes lysine in 0 to 1.5 mol %, histidine in 0.7 to 2.0 mol %, and arginine in 2.0 to 3.0 mol %, with respect to the total substance quantity of all amino acids of the protein. The content of the abovementioned amino acids can be determined as remarked previously.

Moreover, it is especially preferred in the context of this object if the at least one protein has a calcium content of 4 to 400 mg, a potassium content of 50 to 800 mg, a phosphorous content of 1600 to 3200 mg, and a sulfur content of 1300 to 1700 mg, each with respect to 1 kg of the at least one protein. The contents indicated above can be determined by means of atom emission spectrometry (ICP-OES), as set out above.

As regards other preferred embodiments of this object, the remarks made concerning the antiperspirant cosmetic agents according to the invention, the use according to the invention, and the method according to the invention apply mutatis mutandis. Finally, another object of the present invention is a non-therapeutic cosmetic method for preventing and/or reducing body odor triggered by perspiration and or body perspiration per se in which an antiperspirant cosmetic agent according to the invention is applied to the skin, particularly to the skin of the underarms, and remains on the skin of the underarms for at least 1 hour, preferably for at least 2 hours, more preferably for at least 4 hours, particularly for at least 6 hours.

However, a provision can also be made in relation to the method according to the invention that a cosmetic agent including at least one antiperspirant aluminum and/or zirconium halide and/or hydroxy halide is first applied, followed by the cosmetic agent according to the invention. It is also possible, however, to first apply the cosmetic agent according to the invention and then to use a cosmetic agent including at least one antiperspirant aluminum and/or zirconium halide and/or hydroxy halide. Furthermore, the antiperspirant cosmetic agent according to the invention as well as the cosmetic agent including at least one antiperspirant aluminum and/or zirconium halide and/or hydroxy halide can also be applied simultaneously to the skin. The time span between the application of the two agent is from 0 seconds to 24 hours. Moreover, it is preferred if, after application, the agents remain on the skin of the underarms for at least 1 hour, preferably for at least 2 hours, more preferably for at least 4 hours, particularly for at least 6 hours.

As regards the method according to the invention, the remarks made concerning the antiperspirant cosmetic agents according to the invention and the use according to the invention apply mutatis mutandis.

The following examples explain the present invention without limiting it:

### EXAMPLES

1. Change in Light Absorption

The following proteins from Poaceae of the genus Triticum (wheat) were used:
1) Hydrolyzed wheat protein with at least one lauryldimonium hydroxypropyl group and an average molecular weight M_{w} of about 4000 Da, and
2) Hydrolyzed wheat protein with an average molecular weight M_{w} of about 1000 Da, and
3) Hydrolyzed wheat protein with at least one hydroxypropyltrimonium group and an average molecular weight M_{w} of about 3500 Da.

The change in light absorption that is brought about by the abovementioned proteins in a pH range of 4.0 to 8.0 upon a change in pH of at least 0.5 is determined as follows:

TABLE 1
Sample solution (data in wt %)

| | E-I* | E-II* | E-III* |
|---|---|---|---|
| Hydrolyzed wheat protein with at least one | 4.0 | — | — |
| lauryldimonium hydroxypropyl group a) | | | |
| Hydrolyzed wheat protein b) | — | 5.6 | — |
| Hydrolyzed wheat protein with at least one | — | — | 4.4 |
| hydroxypropyltrimonium group c) | | | |
| HCI | Up to | Up to | Up to |
| | pH | pH | pH |
| Water | Up to | Up to | Up to |
| | 100 | 100 | 100 |

*according to the invention
a) Hydrotriticum QM NSP-LQ (WD); (INCI: Cocodimonium hydroxypropyl hydrolyzed wheat protein; 25 wt % solution in water; M_{w} about 4000 Da; Croda)
b) Cropeptid W PF-LQ (WD); (INCI: Hydrolyzed wheat protein and hydrolyzed wheat starch; 18 wt % solution in water; M_{w} about 1000 Da; Croda)
c) Hydrotriticum WQ PE-LQ (WD); (INCI: Hydrolyzed wheat protein; 22.5 wt % solution in water; M_{w} about 3500 Da; Croda)

A Metrohm Titrando 905 by Metrohm (USA), which is equipped with a Metrohm 6.1115.000 optrode and a Metrohm pH electrode, was used to determine the change in light absorption. The Metrohm Titrando 905 was controlled using the software Tiamo by Metrohm. 30 ml of a sample solution according to table 1, which had a pH of 3.0, was first readied in the open sample vessel of the Metrohm Titrando 905. A 1 wt % sodium hydrogen carbonate solution was then added continuously at 23° C. and 1.013 mbar under stirring (stirring speed 8 of the Titrando 905, which corresponds to about 750 to 850 rpm) until a pH of 7.5 was reached. During the addition of the 1 wt % sodium hydrogen carbonate solution, the light transmission of a light beam through this sample solution was measured using a Metrohm 6.1115.000 optrode at a wavelength of 574 nm (greenish yellow) in mV (resolution 0.1 mV). Each measurement was performed twice and the average found.

The change in light absorption that was brought about by the abovementioned peptides was determined according to the formula AL=[(|Li|/|Lo|]*100. In this formula, Lᵢ stands for the after the change in the pH by at least 0.5 in the pH range from 4.0 to 8.0, preferably pH 4.5 to 7.5, particularly pH 5.0 to 7.0. In this formula, L₀ stands for the difference of the light transmissions at pH 4.0 and at pH 8.0, preferably at pH 4.5 and at pH 7.5, particularly at pH 5.0 and at pH 7.0.

Upon a change in pH of 1.0 between pH 5.5 and pH 6.5 (light absorption at pH 6.5 forms value Lᵢ) in a pH range from 4.5 to 7.5 (difference of light absorption at pH 7.5 minus pH 4.5 forms value L₀), these proteins brought about the change in light absorption AL indicated in table 2.

TABLE 2
Change in light absorption AL

| Sample solution | L [%] |
|---|---|
| E-I | 100 |
| E-II | 32 |
| E-III | 41 |

2. In-Vivo Test for Antiperspirant Effect

To determine the antiperspirant effect, an antiperspirant study was performed on the backs of 16 test subjects. The following antiperspirant agents were used for this purpose:

| Antiperspirant agent | No. |
|---|---|
| Aqueous solution with 10% ACH, pH 4 | V-I |
| Aqueous solution with 5%* protein a), pH 2-4 | E-IV** |
| Aqueous solution with 5%* protein b), pH 2-4 | E-V** |
| Aqueous solution with 5%* protein c), pH 2-4 | E-VI** |

*active substance
*according to the invention
a) Hydrotriticum QM NSP-LQ (WD); (INCI: Cocodimonium hydroxypropyl hydrolyzed wheat protein; 25 wt % solution in water; M_{w} about 4000 Da; Croda)
b) Cropeptid W PF-LQ (WD); (INCI: Hydrolyzed wheat protein and hydrolyzed wheat starch; 18 wt % solution in water; M_{w} about 1000 Da; Croda)
c) Hydrotriticum WQ PE-LQ (WD); (INCI: Hydrolyzed wheat protein; 22.5 wt % solution in water; M_{w} about 3500 Da; Croda)

40 μl of the antiperspirant agent V-1 and 75 μl of the antiperspirant cosmetic agents according to the invention E-IV, E-V and E-Vl were applied to the backs of 16 test subjects on one side of the backbone. After 5 minutes, the treated areas were covered with occlusive non-adsorbent film. After 2 hours, these non-adsorbent pads were removed. The compositions were applied to the backs of the test subjects in the manner described above on four successive days. 24 hours after the last application of the composition, absorbent pads were placed on the test subjects' backs in the areas in which the compositions had been applied previously. In addition, pads were also placed on the other side of the backbone at the same height to serve as a control. After the test subjects had sweat in a sauna for about 15 minutes at 80° C., the amount of sweat absorbed by the pads was measured gravimetrically, and each composition was compared to the respective corresponding untreated area on the back. The reduction in sweat was determined from the gravimetric determination of the amount of sweat, and all of the values found were statistically significant.

The reduction in sweat achieved by the respective composition in comparison to an untreated area of the skin is shown in the following table:

| No. | Reduction in sweat |
|---|---|
| V-1 | 50% |
| E-IV | 8% |
| E-V | 18% |
| E-VI | 1.6% |

The use of the specific protein results in a significant reduction in sweat production and in a satisfactory antiperspirant effect.

3. Formulations:

The protein from Poaceae of the genus Triticum (wheat) used in the following examples is preferably selected from among the following proteins:
1) Hydrolyzed wheat protein with at least one lauryldimonium hydroxypropyl group and an average molecular weight M_{w} of about 4000 Da, and
2) Hydrolyzed wheat protein with an average molecular weight M_{w} of about 1000 Da, and
3) Hydrolyzed wheat protein with at least one hydroxypropyltrimonium group and an average molecular weight M_{w} of about 3500 Da.

Antiperspirant cosmetic agents according to the invention with a pH of 2.5 to 10.0 (indicated quantities in wt %)

| | 1 | 2 | 3 | 4 | 5 | 6 | 7 |
|---|---|---|---|---|---|---|---|
| Isopropyl myristate | 0.50 | 0.10 | 0.50 | 1.0 | 2.0 | 3.0 | 5.0 |
| Protein | 0.50 | 2.0 | 3.0 | 5.0 | 7.0 | 10 | 20 |
| Eumulgin B3 d) | 3.0 | 3.0 | 3.0 | 4.0 | 4.0 | 4.0 | 5.0 |
| Perfume | 0.10 | 0.20 | 0.30 | 0.30 | 0.50 | 0.8 | 1.0 |
| Preservative | 0.50 | 0.50 | 0.50 | 0.80 | 0.80 | 1.5 | 2.0 |
| pH adjuster | Up to | Up to | Up to | Up to | Up to | Up to | Up to |
| | pH | pH | pH | pH | pH | pH | pH |
| Water | Up to | Up to | Up to | Up to | Up to | Up to | Up to |
| | 100 | 100 | 100 | 100 | 100 | 100 | 100 |

d) Eumulgin 83 (INCI: Ceteareth-30; BASF)

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant cosmetic agent, comprising:
a) at least one substance selected from the group of cosmetic oils that are liquid at 20° C. and 1013 hPa, odorants and waxes,
b) propellant in a total quantity of 0 to 99 wt % with respect to the total weight of the antiperspirant cosmetic agent, and
c) at least one protein in a total quantity of 0.1 to 70 wt % with respect to the total weight of the antiperspirant cosmetic agent, with the at least one protein occurring in Poaceae of the genus Triticum and/or Oryza and/or Avena and with the at least one protein bringing about a change in the light absorption of 1 to 100% when the pH changes from at least 0.5 to a pH range of 4.0 to pH 8.0, a temperature of 20° C. to 40° C. and a concentration of the protein of 0.001 to 10 wt %, with respect to the total weight of the sample mixture used to determine the change in light absorption,
with the antiperspirant cosmetic agent not including any aluminum and/or zirconium halides and/or hydroxy halides.

2. The antiperspirant cosmetic agent as set forth in claim 1, wherein the at least one protein is included in a total quantity of 0.5 to 60 wt % based on the total weight of the antiperspirant cosmetic agent.

3. The antiperspirant cosmetic agent as set forth in claim 1, wherein the at least one protein is included in a total quantity of 2 to 40 wt % based on the total weight of the antiperspirant cosmetic agent.

4. The antiperspirant cosmetic agent as set forth in claim 1, wherein the at least one protein is included in a total quantity of 2.5 to 30 wt % based on the total weight of the antiperspirant cosmetic agent.

5. The antiperspirant cosmetic agent as set forth in claim 1, wherein the at least one protein is included in a total quantity of 3 to 20 wt % based on the total weight of the antiperspirant cosmetic agent.

6. The antiperspirant cosmetic agent as set forth in claim 1, wherein the at least one protein has an average molecular weight Mw of 150 to 100,000 Da.

7. The antiperspirant cosmetic agent as set forth in claim 1, wherein the at least one protein has an average molecular weight Mw of 180 to 50,000 Da.

8. The antiperspirant cosmetic agent as set forth in claim 1, wherein the at least one protein has an average molecular weight Mw of 200 to 10,000 Da.

9. The antiperspirant cosmetic agent as set forth in claim 1, wherein the at least one protein has an average molecular weight Mw of 250 to 8,000 Da.

10. The antiperspirant cosmetic agent as set forth in claim 1, wherein the at least one protein has an average molecular weight Mw of 300 to 5,000 Da.

11. The antiperspirant cosmetic agent as set forth in claim 1, wherein the at least one protein brings about a change in the light absorption upon a change in pH of at least 0.5 in a pH range from pH 4.5 to pH 7.5, with a concentration of 0.001 to 10 wt % protein, with respect to the total weight of the sample mixture used for the pH measurement, and a temperature of 20° C.

12. The antiperspirant cosmetic agent as set forth in claim 1, wherein the change in pH is achieved through the addition of hydrogen carbonates or carbonates.

13. The antiperspirant cosmetic agent as set forth in claim 1, wherein the at least one protein is selected from the group of (i) non-modified proteins; (ii) hydrolyzed proteins; (iii) chemically modified proteins; (iv) physically modified proteins; (v) hydrolyzed non-modified proteins; (vi) hydrolyzed and chemically modified proteins; (vii) hydrolyzed and physically modified proteins; as well as (viii) mixtures thereof.

14. The antiperspirant cosmetic agent as set forth in claim 13, wherein the at least one protein is a cationically modified protein having one or more residue(s) of the formula R¹—N⁺(CH₃)₂—CH₂—CH(OH)—CH₂—X—R, in which R stands for an alkyl group with 1 to 30 carbon atoms, an alkenyl group with 1 to 30 carbon atoms, a hydroxyalkyl group with 1 to 30 carbon atoms, a C₁₀₋₁₈ alkyl or a C₁₀₋₁₈ alkenyl group, X stands for O, N or S, and R stands for the protein residue.

15. The antiperspirant cosmetic agent as set forth in claim 1, wherein the at least one protein is a protein isolated from Poaceae of the genus Triticum (wheat), with the protein being hydrolyzed and/or with the protein being functionalized with at least one cationic group selected from lauryldimonium hydroxypropyl groups, hydroxypropyltrimonium groups, as well as mixtures thereof.

16. The antiperspirant cosmetic agent as set forth in claim 15, wherein the at least one protein includes lysine in 0 to 1.5 mol %, histidine in 0.7 to 2.0 mol %, and arginine in 2.0 to 3.0 mol %, with respect to the total substance quantity of all amino acids of the protein.

17. The antiperspirant cosmetic agent as set forth in claim 15, wherein the at least one protein has a calcium content of 4 to 400 mg, a potassium content of 50 to 800 mg, a phosphorous content of 1600 to 3200 mg, and a sulfur content of 1300 to 1700 mg, each with respect to 1 kg of the at least one protein.

18. A non-therapeutic cosmetic method for preventing and/or reducing body perspiration, comprising applying the antiperspirant cosmetic agent of claim 1 and leaving the cosmetic on the skin of the underarms for at least 1 hour.

19. A packaging unit (kit of parts), comprising—packaged separately from one another—
a) at least one first container (C1), including a cosmetic agent (M1) comprising at least one antiperspirant agent, and
b) at least one second container (C2), including a cosmetic agent (M2) comprising at least one protein, with the at least one protein occurring in Poaceae of the genus Triticum and/or Oryza and/or Avena, with the at least one protein bringing about a change in the light absorption of 1 to 100% when the pH changes from at least 0.5 to a pH range of 4.0 to pH 8.0, a temperature of 20° C. to 40° C. and a concentration of the protein of 0.001 to 10 wt %, with respect to the total weight of the sample mixture used to determine the change in light absorption, and with the cosmetic agent (M2) including no aluminum and/or zirconium halides and/or hydroxy halides.

20. An antiperspirant cosmetic agent, including
a) at least one substance selected from the group of cosmetic oils that are liquid at 20° C. and 1013 hPa, odorants and waxes,
b) propellant in a total quantity of 0 to 99 wt % with respect to the total weight of the antiperspirant cosmetic agent, and
c) at least one protein isolated from Poaceae of the genus Triticum (wheat) in a total quantity of 0.1 to 70 wt % with respect to the total weight of the antiperspirant cosmetic agent, with the protein being hydrolyzed and/or with the protein being functionalized with at least one cationic group selected from lauryldimonium hydroxypropyl groups, hydroxypropyltrimonium groups and mixtures thereof,
with the antiperspirant cosmetic agent not including any aluminum and/or zirconium halides and/or hydroxy halides.
